(19) United States
(12) Patent Application Publication
(10) Pub. No.: US 2024/0075148 A1
(43) Pub. Date: Mar. 7, 2024

(54) POLY-ION COMPLEX MICELLE

(71) Applicant: KAWASAKI INSTITUTE OF INDUSTRIAL PROMOTION, Kawasaki-shi, Kanagawa (JP)

(72) Inventors: Kazunori KATAOKA, Kawasaki-shi (JP); West Kristian Dizon PARAISO, Kawasaki-shi (JP); Sabina QUADER, Kawasaki-shi (JP); Shigeto FUKUSHIMA, Kawasaki-shi (JP)

(21) Appl. No.: 18/259,327

(22) PCT Filed: Dec. 28, 2021
(86) PCT No.: PCT/JP2021/049002

§ 371 Date: Jun. 26, 2023

Related U.S. Application Data
(60) Provisional application No. 63/130,861, filed on Dec. 28, 2020.

(51) Int. Cl. A61K 47/42; A61K 9/107; C08G 65/332; C08G 81/00

(52) U.S. Cl. CPC A61K 47/42; A61K 9/1075; C08G 65/332; C08G 81/00

(57) ABSTRACT

A poly-ion complex micelle comprising: a block copolymer having a hydrophilic block portion, a cationic hydrophobic block portion and a crosslinking block portion positioned between the hydrophilic block and the cationic hydrophobic block, and an anionic molecule drug encapsulated by the block copolymer, wherein the crosslinking block has a hydrazone bond, the block copolymer comprises the first block copolymer chain and the second block copolymer chain, the first block copolymer chain and the second block copolymer chain crosslinked to each other in the crosslinking block portion, the hydrophilic block portion comprises the first hydrophilic block of the first block copolymer chain and the second hydrophilic block of the second block copolymer chain, and the cationic hydrophobic block portion comprises the first cationic hydrophobic block of the first block copolymer chain.

The present application claims priority based on U.S. Provisional Patent Application No. 63/130,861 filed on Dec. 28, 2020, and the contents of which are incorporated herein by reference.

### TECHNICAL FIELD

The present invention relating to the poly-ion complex micelle.

### BACKGROUND ART

Conventionally, poly-ion complex micelles (hereafter, sometimes referred to as “PIC micelles”) have been used for nucleic acids (e.g., pDNA, mRNA, siRNA, ASO), and negatively-charged, large molecules that has ionic interactions with cationic polymers (e.g., PEG-polylysine, PEG-poly-aspartate(diethylenetriamine)). For example, Non-Patent Literatures 1 and 2 describe a poly-ion complex micelle including a poly(ethylene glycol)-poly(lysine) diblock copolymer and an anionic drug encapsulated by the diblock copolymer.

### CITATION LIST

### Non Patent Literature

NPL 1: Wang C, Chen Q, Wang Z, Zhang X. An enzyme-responsive polymeric super-amphiphile. Angew. Chemie-Int. Ed. 49(46), 8612-8615 (2010).
NPL 2: H. S. Min, H. J. Kim, M. Naito, S. Ogura, K. Toh, K. Hayashi, B. S. Kim, S. Fukushima, Y. Anraku, K. Miyata, K. Kataoka. Systemic brain delivery of antisense oligonucleotides across the blood-brain barrier with a glucose-installed polymeric nanocarrier. Angew. Chem. Int. Ed. 59 (21), 8173-8180 (2020)

### SUMMARY OF INVENTION

### Technical Problem

In a conventional poly-ion complex micelles as described in Non-Patent Literature 1, simple ionic interaction between the drug and the polymer causes formation of a poly-ion complex micelles which have a hydrophobic inner core and an outer layer of PEG. However, conventional poly-ion complex micelles easily collapses at physiological conditions because of the disruption of the polymer-drug ionic interaction. Further, free polymer causes non-selective cytotoxicity when liberated. Therefore, small, negatively-charged molecules were challenging to encapsulate because they are water-soluble and would easily leak out of the delivery system, leading to premature release.

The present invention has been made in consideration of the above-described circumstances, and an object thereof is to provide a poly-ion complex micelle which is capable of encapsulating negatively-charged molecules stably.

### Solution to Problem

In order to solve the above-described problems, the present invention employs the following configurations.
(1) A poly-ion complex micelle including:
   a block copolymer having a hydrophilic block portion, a cationic hydrophobic block portion and a crosslinking block portion positioned between the hydrophilic block portion and the cationic hydrophobic block portion, and
   an anionic molecule drug encapsulated by the block copolymer,
   wherein the crosslinking block portion has a hydrazone bond,
   the block copolymer includes the first block copolymer chain and the second block copolymer chain, the first block copolymer chain and the second block copolymer chain crosslinked to each other in the crosslinking block portion,
   the hydrophilic block portion includes the first hydrophilic block of the first block copolymer chain and the second hydrophilic block of the second block copolymer chain, and
   the cationic hydrophobic block portion includes the first cationic hydrophobic block of the first block copolymer chain.
(2) The poly-ion complex micelle according to (1) above, wherein the cationic hydrophobic block portion further includes the second cationic hydrophobic block of the second block copolymer chain.
(3) The poly-ion complex micelle according to (2) above, wherein the block copolymer is represented by Formula (I):

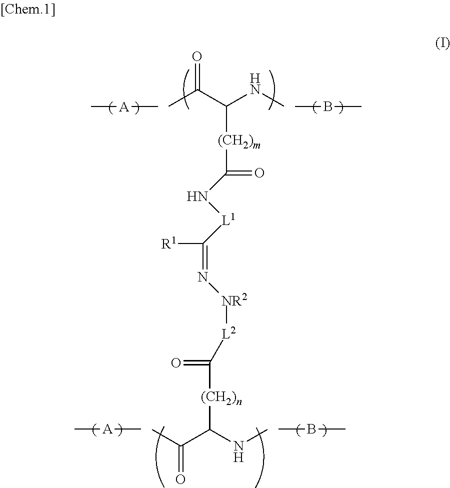

wherein A represents a repeating unit which constitutes the first hydrophilic block or the second hydrophilic block; B represents a repeating unit which constitutes the first cationic hydrophobic block or the second cationic hydrophobic block; m represents 1 or 2; L¹ represents a divalent linking group; R¹ represents a hydrogen atom, an aliphatic hydrocarbon group, or an aromatic hydrocarbon group; R² represents a hydrogen atom or a methyl group; L² represents a single bond or a divalent linking group; and n represents 1 or 2.
(4) The poly-ion complex micelle according to (1) above, wherein the block copolymer is represented by Formula (II):

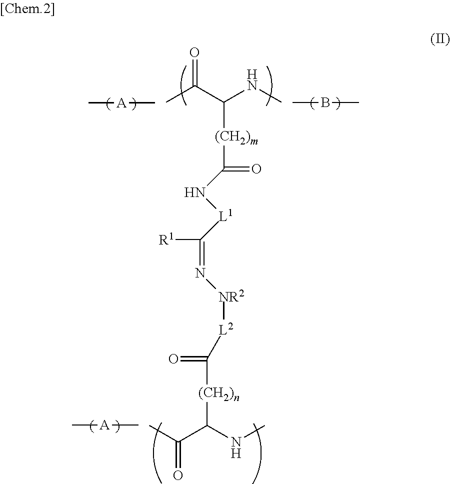

wherein A represents a repeating unit which constitutes the first hydrophilic block or the second hydrophilic block; B represents a repeating unit which constitutes the first cationic hydrophobic block; m represents 1 or 2; L¹ represents a divalent linking group; R¹ represents a hydrogen atom, an aliphatic hydrocarbon group, or an aromatic hydrocarbon group; R² represents a hydrogen atom or a methyl group; L² represents a single bond or a divalent linking group; and n represents 1 or 2.
(5)The poly-ion complex micelle according to any one of (1) to (4) above, wherein the first cationic hydrophobic block is constituted of a repeating structure derived from polylysine.
(6)The poly-ion complex micelle according to (2) or (3) above, wherein the second cationic hydrophobic block is constituted of a repeating structure derived from polylysine.
(7) The poly-ion complex micelle according to any one of (1) to (6) above, which has a particle size of 20 to 100 nm, and a polydispersity index of 0.05 to 0.3.
(8) The poly-ion complex micelle according to any one of (1) to (7) above, wherein the anionic molecule drug has a net negative charge of −25 to −1 at physiological pH.
(9) The poly-ion complex micelle according to any one of(1) to (8) above, wherein the anionic molecule drug is a nucleic acid drug.

### Advantageous Effects of Invention

According to the present invention, it is possible to provide a poly-ion complex micelle which is capable of encapsulating negatively-charged molecules stably. In particular, the present invention may provide a poly-ion complex micelle useful for encapsulating small, negatively-charged molecules.

### BRIEF DESCRIPTION OF DRAWINGS

FIG. 1 is a schematic diagram showing one embodiment of the poly-ion complex micelle according to the present invention.

FIG. 2 is a schematic diagram showing one embodiment of the poly-ion complex micelle according to the present invention.

FIG. 3 shows the results of the cellular uptake experiments using RPMI 2650.

FIG. 4 shows the results of the cellular uptake experiments using RPMI 2650.

FIG. 5 is the timeline of culture of RPMI 2650 for the transwell permeability experiments.

FIG. 6 shows the results of the transwell permeability experiments.

FIG. 7 is a schematic diagram showing the non-poly-ion complex micelle prepared for the cellular uptake experiments using brain cells.

FIG. 8 shows the results of the cellular uptake experiments using KT-5 (astrocytes).

FIG. 9 shows the results of the cellular uptake experiments using BV-2 (microglia).

FIG. 10 shows the results of the cellular uptake experiments using GT1-7-5 (neurons).

FIG. 11 shows the results of the cellular uptake experiments using rat primary brain endothelial cells.

### DESCRIPTION OF EMBODIMENTS

<Poly-Ion Complex Micelle>

The poly-ion complex micelle according to the present embodiment includes a block copolymer having a hydrophilic block portion, a cationic hydrophobic block portion and a crosslinking block portion positioned between the hydrophilic block and the cationic hydrophobic block, and an anionic molecule drug encapsulated by the block copolymer. The crosslinking block portion has a hydrazone bond. The block copolymer includes the first block copolymer chain and the second block copolymer chain. The first block copolymer chain and the second block copolymer chain are crosslinked to each other in the crosslinking block portion. The hydrophilic block portion includes the first hydrophilic block of the first block copolymer chain and the second hydrophilic block of the second block copolymer chain. The cationic hydrophobic block portion includes the first cationic hydrophobic block of the first block copolymer chain.

### First Embodiment

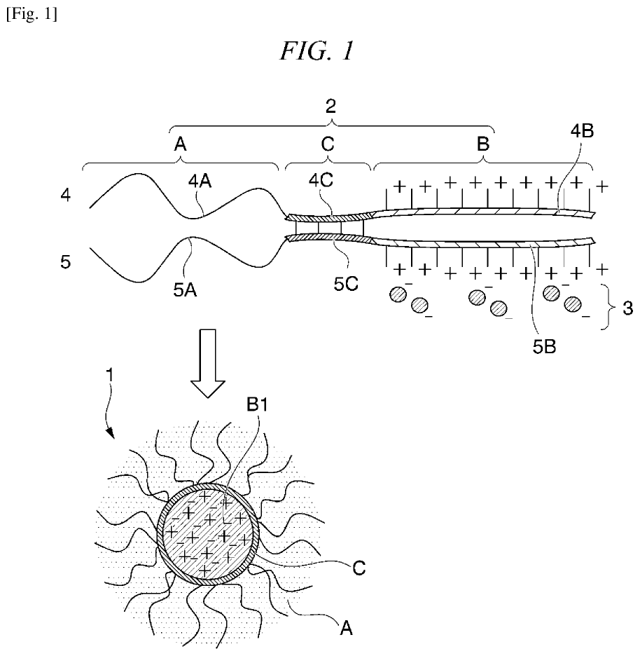

FIG. 1 is a schematic diagram showing one embodiment of the poly-ion complex micelle according to the present invention. As shown in FIG. 1, the poly-ion complex micelle 1 is formed by self-assembly of the block copolymer 2 and the anionic molecule drug 3. Specifically, ionic interaction between the cationic hydrophobic block portion B and the anionic molecule drug 3 to form a core B1 loaded with the anionic molecule drug 3. The crosslinking block portion C surrounds the core B1 to stabilize the core B1. The hydrophilic block portion A forms a shell, thus forming the poly-ion complex micelle 1.

By virtue of the crosslinking block C portion surrounding the core B1, the anionic molecule drug 3 can be prevented from leaking out, unless there are physiological triggers like low endosomal pH inside the cell.

The block copolymer 2 is composed of the first block copolymer chain 4 and the second block copolymer chain 5. The first block copolymer chain 4 includes the firth hydrophilic block 4A, the first crosslinking block 4C, and the first cationic hydrophobic block 4B, in this order. The second block copolymer chain 5 includes the second hydrophilic block 5A, the second crosslinking block 5C, and the second cationic hydrophobic block 5B, in this order. The block copolymer 2 is formed by crosslinking between the first crosslinking block 4C of the first block copolymer chain 4 and the second crosslinking block 5C of the second block copolymer chain 5. The hydrophilic block portion A includes the first hydrophilic block 4A and the second hydrophilic block 5A. The hydrophobic block portion B includes the first hydrophobic block 4B and the second hydrophobic block 5B.

The poly-ion complex micelle according to the present embodiment preferably has a particle size of 20 to 100 nm, more preferably 35 to 50 nm. Further, the poly-ion complex micelle preferably has a polydispersity index of 0.05 to 0.3, more preferably 0.05 to 0.1.

(Block Copolymer)

In the block copolymer, “hydrophilicity” and “hydrophobicity” of the hydrophilic block and the hydrophobic block are relative. The “hydrophilicity” and “hydrophobicity” of the hydrophilic block and the hydrophobic block may be defined by log P values. The log P value is the logarithm of the octanol/water partition co-efficient (Pow) and is an effective parameter that can characterize its hydrophilicity/hydrophobicity for a wide range of compounds. It means that the hydrophobicity increases when the log P value is greater than 0 and increases toward the plus side, and the hydrophilicity increases when the log P value increases toward the minus side.

Each of the first hydrophilic block and the first hydrophobic block may have one kind of repeating unit or two or more kinds of repeating units.

Each of the second hydrophilic block and the second hydrophobic block may have one kind of repeating unit or two or more kinds of repeating units.

Hereinafter, the first hydrophilic block and the second hydrophilic block may be collectively referred to as “the hydrophilic block”, and the first hydrophobic block and the second hydrophobic block may be collectively referred to as “the hydrophobic block”

The number of repeating units and the molecular weight of the hydrophilic block may be appropriately controlled according to the molecular weight of the anionic molecular drug.

The number of repeating units of the hydrophilic block may be, for example, 1 or more, 5 or more, 10 or more, 20 or more, or 45 or more. Further, the number of repeating units of the hydrophilic block may be, for example, 1000 or less, 700 or less, or 450 or less.

The molecular weight of the hydrophilic block may be, for example, 1,000 Da or more, 2,000 Da or more, or 5,000 Da or more. The molecular weight of the hydrophilic block may be, for example, 40,000 Da or less, 30,000 Da or less, or 20,000 Da or less.

The number of repeating units and the molecular weight of the hydrophobic block may be appropriately controlled according to the molecular weight of the anionic molecular drug.

The number of repeating units of the hydrophobic block may be, for example, 5 or more, 10 or more, or 20 or more. The number of repeating units of the hydrophobic block may be, for example, 1000 or less, 800, or less, 600, or less, 500, or less, 300, or less, 200 or less, 100 or less, or 60 or less.

The molecular weight of the hydrophobic block may be, for example, 1,000 Da or more, 2,000 Da or more, 3,000 Da or more, or 5,000 Da or more. The molecular weight of the hydrophobic block may be, for example, 50,000 Da or less, 30,000 Da or less, 16,000 Da or less, or 10,000 Da or less.

Specific examples of the hydrophilic block include a block having at least one

repeating unit selected from the group consisting of a repeating unit derived from polyethylene glycol, a repeating unit derived from poly(ethylethylenephosphate), a repeating unit derived from polyvinyl alcohol, a repeating unit derived from polyvinylpyrrolidone, a repeating unit derived from poly(oxazoline), and a repeating unit derived from poly(N-(2-hydroxypropyl)methacrylamide) (PHPMA). Among these examples, as the hydrophilic block, a block having a repeating unit derived from polyethylene glycol is preferable.

Examples of the hydrophobic block include a block having at least one repeating unit selected from the group consisting of repeating units derived from amino acids and derivatives thereof, preferably repeating units derived from polyamino acids and derivatives thereof. Examples of polyamino acids include polylysine, polyornithine, poly(2,6-diaminoheptanoic acid), poly(2,8-diaminooctanoic acid), poly(2,9-diaminononanoic acid), polyarginine and polyhistidine. Examples of amino acid derivatives include poly[N-(2-aminoethyl)aspartamide](PAsp-(EDA)), poly{N-[N′-(2-aminoethyl)-2-aminoethyl]aspartamide} (PAsp(DET)), poly(N-{N′-[N″-(2-aminoethyl)-2-aminoethyl]-2-aminoethyl}aspartamide) (PAsp(TET)), and poly[N-(N′-{N″-[N′″-(2-aminoethyl)-2-aminoethyl]-2-aminoethyl}-2-aminoethyl)aspartamide] (PAsp(TEP)).

Among these examples, the hydrophobic block is preferably constituted of a repeating structure derived from polylysine.

The first hydrophilic block of the first block copolymer chain and the second hydrophilic block of the second block copolymer chain may be the same or different.

The number of the repeating units in the first hydrophilic block of the first block copolymer chain and the number of the repeating units in the second hydrophilic block of the second block copolymer chain may be the same or different.

The first hydrophobic block of the first block copolymer chain and the second hydrophobic block of the second block copolymer chain may be the same or different.

The number of the repeating units in the first hydrophobic block of the first block copolymer chain and the number of the repeating units in the second hydrophobic block of the second block copolymer chain may be the same or different.

The crosslinking block portion is not particularly limited as long as it has a hydrazone bond. The crosslinking block portion may be composed of the first crosslinking block and the second crosslinking block that are crosslinked to each other. Linking groups that crosslink the first crosslinking block and the second crosslinking block has a hydrazone bond. Hereinafter, the first crosslinking block and the second crosslinking block may be collectively referred to as “the crosslinking block”.

Typically, the crosslinking block portion has a repeating unit in which a repeating unit of the first crosslinking block and a repeating unit of the second crosslinking block are linked by a hydrazone bond. Examples of the amino acids and derivatives thereof include aspartic acid, glutamic acid, lysine, ornithine, benzylaspartic acid, benzylglutamic acid, and their derivatives. The crosslinking block may be composed of a polyamino acid or derivative thereof, such as polyaspartic acid, polyglutamic acid, polylysine, polyornithine, poly(benzylaspartic acid), and poly(benzylglutamic acid).

Hereinafter, a repeating unit in which a repeating unit of the first crosslinking block and a repeating unit of the second crosslinking block are linked by a hydrazone bond may be referred to as “the repeating unit (c1)”. A repeating unit of the first crosslinking block which is crosslinked to a repeating unit of the second crosslinking block may be referred to as “the repeating unit (c1-1)”. A repeating unit of the second crosslinking block which is crosslinked to a repeating unit of the first crosslinking block may be referred to as “the repeating unit (c1-2)”.

In one embodiment, the repeating unit (c1) is preferably represented by Formula (c1). The crosslinking block portion may have one kind of the repeating unit (c1) or two or more kinds of the repeating units (c1).

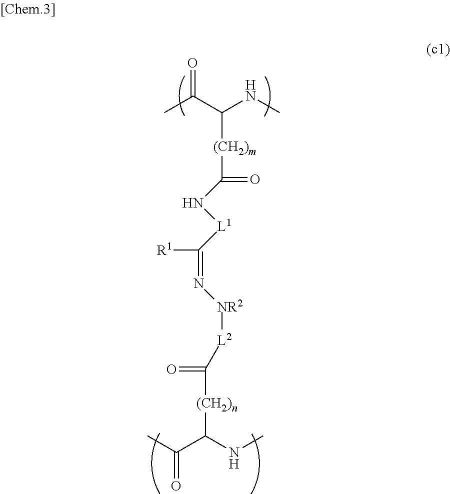

wherein m represents 1 or 2; L¹ represents a divalent linking group; R¹ represents a hydrogen atom, an aliphatic hydrocarbon group, or an aromatic hydrocarbon group; R² represents a hydrogen atom or a methyl group; L² represents a single bond or a divalent linking group; and n represents 1 or 2.

L¹, R¹, R², L², m and n are the same as defined for L¹, R¹, R², L², m and n in Formula (I) below.

The first crosslinking block may have a repeating unit (c2-1) that is not

crosslinked to a repeating unit of the second crosslinking block in addition to the repeating unit (c1-1). The second crosslinking block may have a repeating unit (c2-2) that is not crosslinked to a repeating unit of the first crosslinking block in addition to the repeating unit (c1-2). The repeating unit (c2-1) and the repeating unit (c2-2) may be derived from an amino acid or derivative thereof. Examples of the amino acids and derivatives thereof includes the same as those described above. The first crosslinking block may have one kind of the repeating unit (c2-1) or two or more kinds of the repeating units (c2-1). The second crosslinking block may have one kind of the repeating unit (c2-2) or two or more kinds of the repeating units (c2-2).

The proportion of the repeating unit (c1-1) to the all repeating units constituting the first crosslinking block may be 30 mole % or more, 40 mole % or more, 50 mole % or more, 60 mole % or more. The proportion of the repeating unit (c1-2) to the all repeating units constituting the second crosslinking block may be 30 mole % or more, 40 mole % or more, 50 mole % or more, 60 mole % or more.

In the present embodiment, the block copolymer is preferably represented by Formula (I).

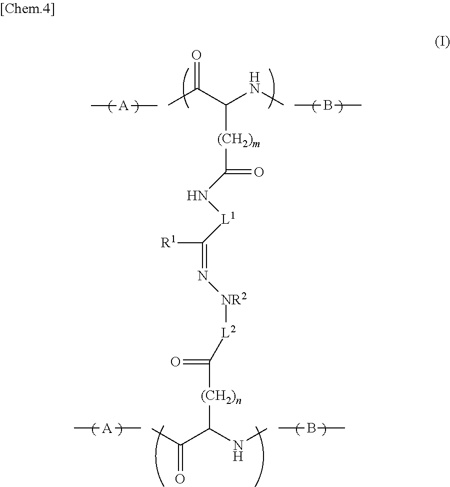

wherein A represents a repeating unit which constitutes the first hydrophilic block or the second hydrophilic block; B represents a repeating unit which constitutes the first cationic hydrophobic block or the second cationic hydrophobic block; m represents 1 or 2; L¹ represents a divalent linking group; R¹ represents a hydrogen atom, an aliphatic hydrocarbon group, or an aromatic hydrocarbon group; R² represents a hydrogen atom or a methyl group; L² represents a single bond or a divalent linking group; and n represents 1 or 2.

In Formula (I), A represents a repeating unit which constitutes the hydrophilic block, and the same repeating units as those described above for the hydrophilic block may be employed.

In Formula (I), B represents a repeating unit which constitutes the cationic hydrophobic block, and the same repeating units as those described above for the hydrophobic block may be employed.

m represents 1 or 2, preferably 1.

n represents 1 or 2, preferably 1.

In Formula (I), L¹ represents a divalent linking group. The divalent linking group is not particularly limited, and preferable examples thereof include a divalent hydrocarbon group which may have a substituent and a divalent linking group containing a hetero atom.

In the case where L¹ is a divalent linking group which may have a substituent, the hydrocarbon group may be either an aliphatic hydrocarbon group or an aromatic hydrocarbon group.

Examples of the aliphatic hydrocarbon group for L¹ include a linear or branched aliphatic hydrocarbon group, and an aliphatic hydrocarbon group containing a ring in the structure thereof.

The linear or branched aliphatic hydrocarbon group preferably has 1 to 10 carbon atoms, more preferably 1 to 6, still more preferably 1 to 4, and most preferably 1 to 3.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—] and a pentamethylene group [—(CH₂)₅—].

As the branched aliphatic hydrocarbon group, branched alkylene groups are preferred, and specific examples include various alkylalkylene groups, including alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂—, and —CH₂CH(CH₃)CH₂CH₂—. As the alkyl group within the alkylalkylene group, a linear alkyl group of 1 to 5 carbon atoms is preferable.

The linear or branched aliphatic hydrocarbon group may or may not have a substituent. Examples of the substituent include a fluorine atom, a fluorinated alkyl group of 1 to 5 carbon atoms, and a carbonyl group.

As examples of the hydrocarbon group containing a ring in the structure thereof for L¹, a cyclic aliphatic hydrocarbon group containing a hetero atom in the ring structure thereof and may have a substituent (a group in which two hydrogen atoms have been removed from an aliphatic hydrocarbon ring), a group in which the cyclic aliphatic hydrocarbon group is bonded to the terminal of the aforementioned chain-like aliphatic hydrocarbon group, and a group in which the cyclic aliphatic group is interposed within the aforementioned linear or branched aliphatic hydrocarbon group, can be given. As the linear or branched aliphatic hydrocarbon group, the same groups as those described above can be used.

The cyclic aliphatic hydrocarbon group preferably has 3 to 20 carbon atoms, and more preferably 3 to 12 carbon atoms.

The cyclic aliphatic hydrocarbon group may or may not have a substituent. Examples of the substituent include an alkyl group, an alkoxy group, a halogen atom, a halogenated alkyl group, a hydroxyl group and a carbonyl group.

aromatic hydrocarbon group for L¹ is a hydrocarbon group having at least one aromatic ring.

The aromatic ring is not particularly limited, as long as it is a cyclic conjugated compound having (4n+2)π electrons, and may be either monocyclic or polycyclic. The aromatic ring preferably has 5 to 30 carbon atoms, more preferably 5 to 20, still more preferably 6 to 15, and most preferably 6 to 12. Here, the number of carbon atoms within a substituent(s) is not included in the number of carbon atoms of the aromatic hydrocarbon group. Examples of the aromatic ring include aromatic hydrocarbon rings, such as benzene, naphthalene, anthracene and phenanthrene; and aromatic hetero rings in which part of the carbon atoms constituting the aforementioned aromatic hydrocarbon rings has been substituted with a hetero atom. Examples of the hetero atom within the aromatic hetero rings include an oxygen atom, a sulfur atom and a nitrogen atom.

Specific examples of the aromatic hetero ring include a pyridine ring and a thiophene ring.

Specific examples of the aromatic hydrocarbon group include a group in which two hydrogen atoms have been removed from the aforementioned aromatic hydrocarbon ring or aromatic hetero ring (arylene group or heteroarylene group); a group in which two hydrogen atoms have been removed from an aromatic compound having two or more aromatic rings (biphenyl, fluorene or the like); and a group in which one hydrogen atom of the aforementioned aromatic hydrocarbon ring or aromatic hetero ring has been substituted with an alkylene group (a group in which one hydrogen atom has been removed from the aryl group within the aforementioned arylalkyl group such as a benzyl group, a phenethyl group, a 1-naphthylmethyl group, a 2-naphthylmethyl group, a 1-naphthylethyl group, or a 2-naphthylethyl group, or a heteroarylalkyl group). The alkylene group which is bonded to the aforementioned aryl group or heteroaryl group preferably has 1 to 4 carbon atoms, more preferably 1 or 2 carbon atoms, and most preferably 1 carbon atom.

With respect to the aromatic hydrocarbon group for L¹, the hydrogen atom within the aromatic hydrocarbon group may be substituted with a substituent. For example, the hydrogen atom bonded to the aromatic ring within the aromatic hydrocarbon group may be substituted with a substituent. Examples of substituents include an alkyl group, an alkoxy group, a halogen atom, a halogenated alkyl group, and a hydroxyl group.

In the case where L¹ represents a divalent linking group containing a hetero atom, preferable examples of the linking group include —O—, —C(═O)—O—, —C(═O)—, —O—C(═O)—O—, —C(═O)—NH—, —NH—, —NH—C(═NH)— (may be substituted with a substituent such as an alkyl group, an acyl group or the like), —S—, —S(═O)₂—, —S(═O)₂—O—, and a group represented by general formula: —Y²¹—O—Y²²—, —Y²¹—O—, —Y²¹—C(═O)—O—, —C(═O)—O—Y²¹—, —[Y²¹—C(═O)—O]_{m″}—Y²², —Y²¹—O—C(═O)—Y²²— or —Y²¹—S(═O)₂—O—Y²²— [in the formulae, Y²¹ and Y²² each independently represents a divalent hydrocarbon group which may have a substituent, O represents an oxygen atom, and m′ represents an integer of 0 to 3].

In the case where the divalent linking group containing a hetero atom is —C(═O)—NH—, —C(═O)—NH—C(═O)—, —NH— or —NH—C(═NH)—, H may be substituted with a substituent such as an alkyl group, an acyl group or the like. The substituent (an alkyl group, an acyl group or the like) preferably has 1 to 10 carbon atoms, more preferably 1 to 8, and most preferably 1 to 5.

In general formulae —Y²¹—O—Y²²—, —Y²¹—O—, —Y²¹—C(═O)—O—, —C(═O)—O—Y²¹—, —[(Y²¹—C(═O)—O]_{m″}—Y²²—, —Y²¹—O—C(═O)—Y²²— or —Y²¹—S(═O)₂—O—Y²²—, Y²¹ and Y²² each independently represents a divalent hydrocarbon group which may have a substituent. Examples of the divalent hydrocarbon group include the same groups as those described above as the “divalent hydrocarbon group which may have a substituent” in the explanation of the aforementioned divalent linking group.

As Y²¹, a linear aliphatic hydrocarbon group is preferable, more preferably a linear alkylene group, still more preferably a linear alkylene group of 1 to 5 carbon atoms, and a methylene group or an ethylene group is particularly desirable.

As Y²², a linear or branched aliphatic hydrocarbon group is preferable, and a methylene group, an ethylene group or an alkylmethylene group is more preferable. The alkyl group within the alkylmethylene group is preferably a linear alkyl group of 1 to 5 carbon atoms, more preferably a linear alkyl group of 1 to 3 carbon atoms, and most preferably a methyl group.

In the group represented by the formula —[Y²¹—C(═O)—O]ₘ″—Y₂₂—, m″ represents an integer of 0 to 3, preferably an integer of 0 to 2, more preferably 0 or 1, and most preferably 1. Namely, it is particularly desirable that the group represented by the formula —[Y²¹—C(═O)—O]ₘ″—Y²²— is a group represented by the formula —Y²¹—C(═O)—O—Y²²—. Among these, a group represented by the formula —(CH₂)_{a′}—C(═O)—O—(CH₂)_{b′}— is preferable. In the formula, a′ is an integer of 1 to 10, preferably an integer of 1 to 8, more preferably an integer of 1 to 5, still more preferably 1 or 2, and most preferably 1. b′ is an integer of 1 to 10, preferably an integer of 1 to 8, more preferably an integer of 1 to 5, still more preferably 1 or 2, and most preferably 1.

In Formula (I), L¹ is preferably a divalent linear or branched hydrocarbon group or a divalent aromatic hydrocarbon group, and more preferably a group in which one hydrogen atom has been removed from a benzyl group.

In Formula (I), R¹ represents a hydrogen atom, an aliphatic hydrocarbon group, or an aromatic hydrocarbon group.

Examples of the aliphatic hydrocarbon group as R¹ include a methyl group, an ethyl group, a propyl group, a butyl group, and a pentyl group. The aliphatic hydrocarbon group as R¹ may have a substituent. Examples of the substituent include a methyl group, an ethyl group, a propyl group, an isopropyl group, a butyl group, an isobutyl group, a tert-butyl group, a pentyl group, an isopentyl group, a tert-pentyl group, a cyclohexyl group, and a trihalomethyl group.

Examples of the aromatic hydrocarbon group as R¹ include a phenyl group, a benzyl group, a pyridyl group, a naphthyl group, a hydroxyphenyl group, a methoxyphenyl group, an ethoxyphenyl group, a xylyl group, a methylphenyl group, a nitrophenyl group, a chlorophenyl group, a fluorophenyl group, an iodophenyl group, and a bromophenyl group.

Among these, R¹ is preferably a hydrogen atom or an aliphatic hydrocarbon group and more preferably a hydrogen atom or a methyl group.

In Formula (I), as the divalent linking group for L², the same groups as those described above for L¹ may be mentioned. In addition, the divalent linking group for L² may be a group represented by the formula -L^{R}-NH—N═C(R¹¹)-L²¹-NH—. In the formula, R¹¹ and L²¹ are the same as defined above for R¹ and L¹ in Formula (I), respectively. L^{R} represents a divalent residual group of a linker. Examples of the linker include a dihydrazide linker, a disulfide linker, an acetal linker and a ketal linker.

Among the above examples, as L², a single bond or a group represented by the formula -L^{R}-NH—N═C(R¹¹)-L²¹-NH— is preferable, and a single bond is more preferable.

(Anionic Molecule Drug)

As used herein, an “anionic molecule drug” refers to a drug molecule having a net negative charge. The anionic molecule drug may be a small-molecule drug, a middle-molecular drug, a high molecular drug, or a nucleic acid drug. Examples of nucleic acid drugs include, but are not limited to, antisense nucleic acids, small interfering nucleic acid (e.g., siRNA), miRNA, mRNA, and plasmid DNA.

The anionic molecule drug preferably has a molecular weight of 20,000 Da or less. The molecular weight of the anionic molecule drug may be 15,000 Da or less, 10,000 Da or less, 8,000 Da or less, 5,000 Da or less, 3,000 Da or less, 2,000 Da or less, or 1,000 Da or less. More specifically, the small molecule drugs may have a molecular weight of 1,000 or less Da. The nucleic acid drugs may have a molecular weight of 20,000 Da or less.

The anionic molecule drug preferably has a net negative charge of −25 to −1 at physiological pH. The physiological pH may be pH 6.5 to 8, preferably pH 7 to 7.5. In one embodiment, the physiological pH may be about pH 7.4.

Examples of anionic molecule drugs include cytarabine triphosphate, gemcitabine triphosphate, fludarabine triphosphate, cladribine triphosphate, capecitabine triphosphate, troxacitabine triphosphate, clofarabine triphosphate, combretastatin A1 diphosphate, adenosine triphosphate, cyclic guanosine monophosphate-adenosine monophosphate, cyclic di-guanosine monophosphate, palmitoyl-coenzyme A, malonyl-coenzyme A. Among these examples, cytarabine triphosphate and gemcitabine triphosphate is preferable.

Specific examples of nucleic acid drugs include, but are limited to, luciferase ASO, fomivirsen, mipomersen, defibrotide, eteplirsen, pegaptinib, nusinersen. Among these examples, luciferase ASO is preferable.

### Second Embodiment

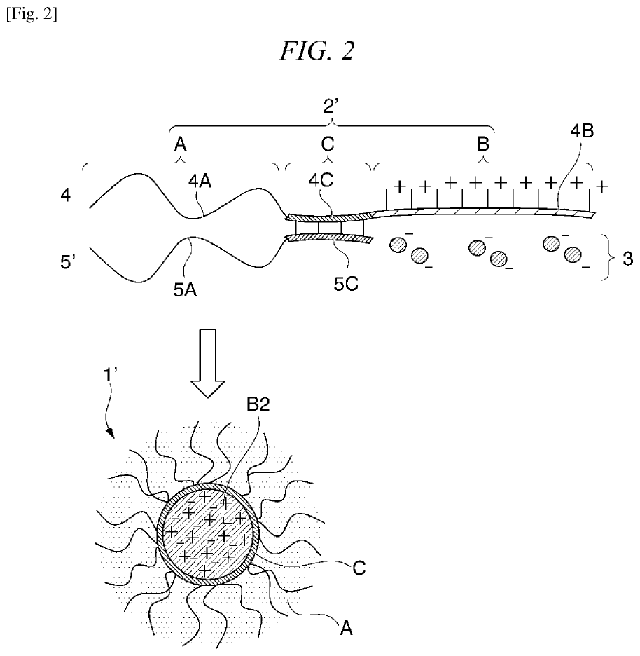

FIG. 2 is a schematic diagram showing another embodiment of the poly-ion complex micelle according to the present invention. As shown in FIG. 2, the poly-ion complex micelle 1′ is formed by self-assembly of the block copolymer 2′ and the anionic molecule drug 3. Specifically, ionic interaction between the cationic hydrophobic block portion B and the anionic molecule drug 3 to form a core B2 loaded with the anionic molecule drug 3. The crosslinking block portion C surrounds the core B2 to stabilize the core B2. The hydrophilic block portion A forms a shell, thus forming the poly-ion complex micelle 1′.

By virtue of the crosslinking block portion C surrounding the core B2, the anionic molecule drug 3 can be prevented from leaking out, unless there are physiological triggers like low endosomal pH inside the cell.

The block copolymer 2′ is composed of the first block copolymer chain 4 and the second block copolymer chain 5′. The first block copolymer chain 4 includes the first hydrophilic block 4A, the first crosslinking block 4C, and the first cationic hydrophobic block 4B, in this order. The second block copolymer chain 5′ includes the second hydrophilic block 5A, and the second crosslinking block 5C. The block copolymer 2′ is formed by crosslinking in the crosslinking block 4C of the first block copolymer chain 4 and the crosslinking block 5C of the second block copolymer chain 5′. The hydrophilic block portion A includes the first hydrophilic block 4A and the first hydrophilic block 5A. The hydrophobic block portion B includes the first hydrophobic block 4B.

The poly-ion complex micelle according to the present embodiment preferably has a particle size of 20 to 100 nm, more preferably 35 to 50 nm. Further, the poly-ion complex micelle preferably has a polydispersity index of 0.05 to 0.3, more preferably 0.05 to 0.15.

(Block Copolymer)

The hydrophilic block portion, the first hydrophilic block, and the second hydrophilic block are the same as those described above.

The crosslinking block portion, the first crosslinking block, and the second crosslinking block are the same as described above.

In the present embodiment, the second block copolymer chain does not include a hydrophobic block. The hydrophobic block portion may be composed of the first hydrophobic block of the first block copolymer chain. The first hydrophobic block is the same as the first hydrophobic block of the first embodiment described above.

The first hydrophilic block of the first block copolymer chain and the second hydrophilic block of the second block copolymer chain may be the same or different.

The number of the repeating units in the first hydrophilic block of the first block copolymer chain and the number of the repeating units in the second hydrophilic block of the second block copolymer chain may be the same or different.

In the present embodiment, the block copolymer is preferably represented by Formula (II).

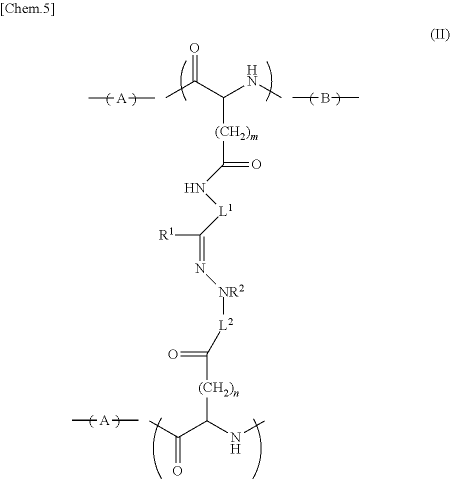

wherein A represents a repeating unit which constitutes the first hydrophilic block or the second hydrophilic block; B represents a repeating unit which constitutes the first cationic hydrophobic block; m represents 1 or 2; L¹ represents a divalent linking group; R¹ represents a hydrogen atom, an aliphatic hydrocarbon group, or an aromatic hydrocarbon group; R² represents a hydrogen atom or a methyl group; L² represents a single bond or a divalent linking group; and n represents 1 or 2.

A, B, m, L¹, R¹, R², L², and n are the same as defined for A, B, m, L¹, R¹, R², L², and n in Formula (I), respectively.

The terminal group of the second crosslinking block is not particularly limited. Examples of the terminal group of the second crosslinking block include, but are not limited to, a hydrogen atom, an acyl group having 1 to 5 carbon atoms (e.g., acetyl group), an amino group, an alkyl group having 1 to 5 carbon atoms (e.g., methyl group), and an alkoxy group having 1 to 5 carbon atoms (e.g., methoxy group).

(Anionic Molecule Drug)

The anionic molecule drug is the same as the anionic molecule drug of the first embodiment described above.

<Method of Producing Poly-Ion Complex Micelle>

### First Embodiment

The poly-ion complex micelle according to the first embodiment may be produced by reacting a compound (Ia-1) represented by Formula (Ia-1) and a compound (Ia-2) represented by Formula (Ia-2) to obtain a block copolymer represented by Formula (I), and allowing a self-assembly of the block copolymer with an anionic molecule drug.

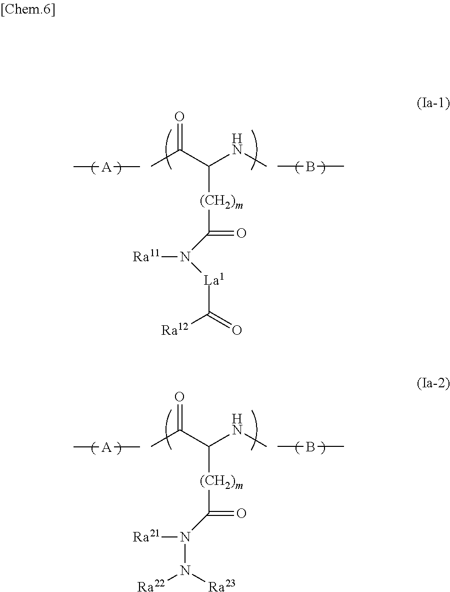

wherein A, B and m are the same as defined for A, B and m in Formula (I); Ra¹¹ represents a hydrogen atom; La¹ represents an alkylene group, an arylene group or an aralkylene group, provided that La¹ may have a substituent which is inactive with a hydrazide group or a hydrazine group; Ra¹² represents a hydrogen atom, an aliphatic hydrocarbon group, or an aromatic hydrocarbon group; Ra²¹ represents a hydrogen atom or a methyl group; and Each of Ra²² and Ra²³ represents a hydrogen atom.

In Formula (Ia-1), Ra¹¹ represents a hydrogen atom.

In Formula (Ia-1), Ra¹² is the same as defined for R¹ in Formula (I).

As the alkylene group, the arylene group or the aralkylene group for La¹, the same alkylene group, arylene group or aralkylene group as described above for the divalent linking group for L¹ in Formula (I) may be selected.

In Formula (Ia-2), Ra²¹ represents a hydrogen atom or a methyl group, preferably a hydrogen atom.

An example of the reaction scheme for producing the poly-ion complex micelle according to the present embodiment is shown below.

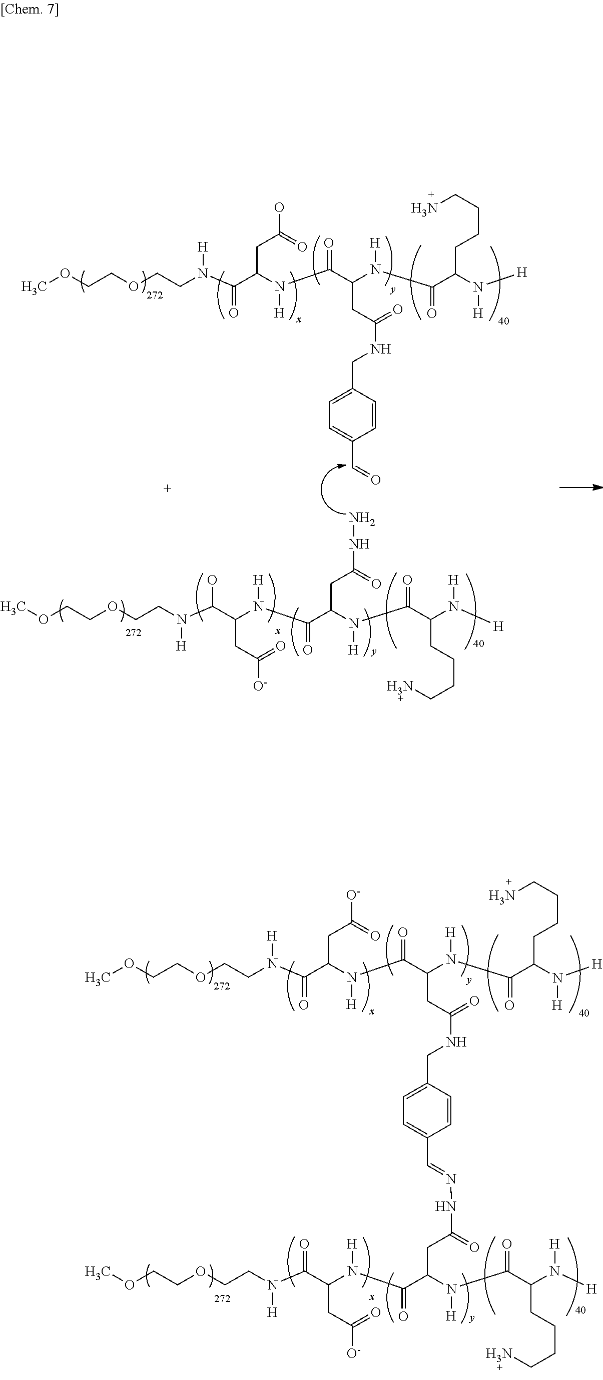

Alternatively, in the case where L² in Formula (I) is a group represented by the formula -L^{R}-NH—N═C(R¹)-L¹-NH—, a block copolymer represented by the following Formula (Ib) may be crosslinked with a linker to obtain the block copolymer represented by Formula (I).

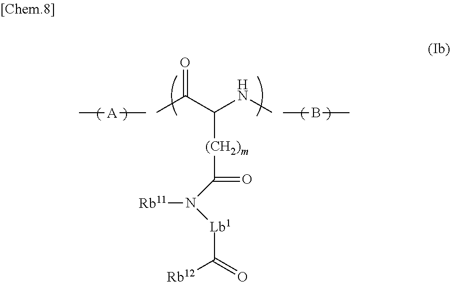

wherein A, B and m are the same as defined for A, B and m in Formula (I); and Lb¹, Rb¹¹ and Rb¹² are the same as defined for La¹, Ra¹¹ and Ra¹² in Formula (Ia-1), respectively.

An example of the alternative reaction scheme for producing the poly-ion complex micelle according to the present embodiment is shown below.

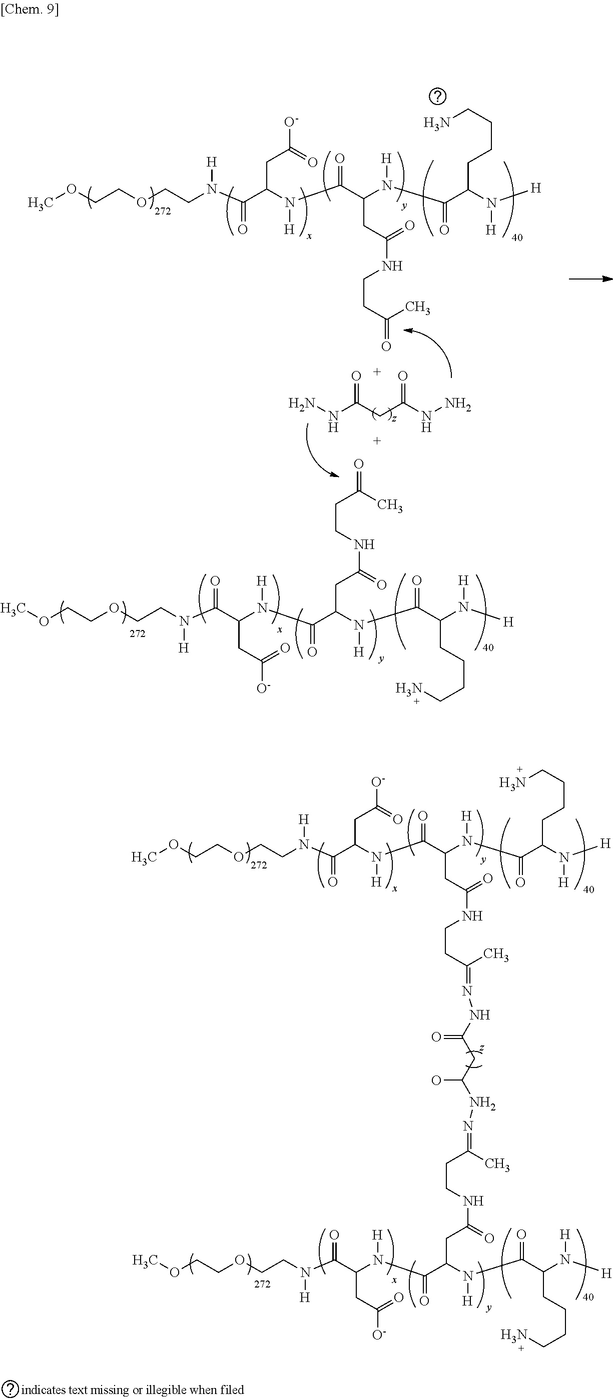

### Second Embodiment

The poly-ion complex micelle according to the second embodiment may be produced by reacting a compound (IIa-1) represented by Formula (IIa-1) and a compound (IIa-2) represented by Formula (IIa-2) to obtain a block copolymer represented by Formula (II), and allowing a self-assembly of the block copolymer with an anionic molecule drug.

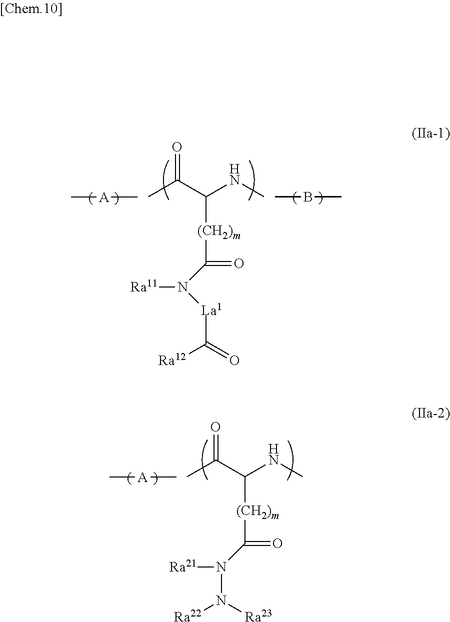

wherein A, B and m are the same as defined for A, B and m in Formula (I); Ra¹¹ represents a hydrogen atom; La¹ represents an alkylene group, an arylene group or an aralkylene group, provided that La¹ may have a substituent which is inactive with a hydrazide group or a hydrazine group; Ra¹² represents a hydrogen atom, an aliphatic hydrocarbon group, or an aromatic hydrocarbon group; Ra²¹ represents a hydrogen atom or a methyl group; and Each of Ra²² and Ra²³ represents a hydrogen atom.

Ra¹¹, Ra¹² and La¹ in Formula (IIa-1) are the same as defined for Ra¹¹, Ra¹² and La¹ in Formula (Ia-1).

Ra²¹, Ra²² and Ra²³ in Formula (IIa-2) are the same as defined for Ra²¹, Ra²² and Ra²³ in Formula (Ia-2).

An example of the reaction scheme for producing the poly-ion complex micelle according to the present embodiment is shown below.

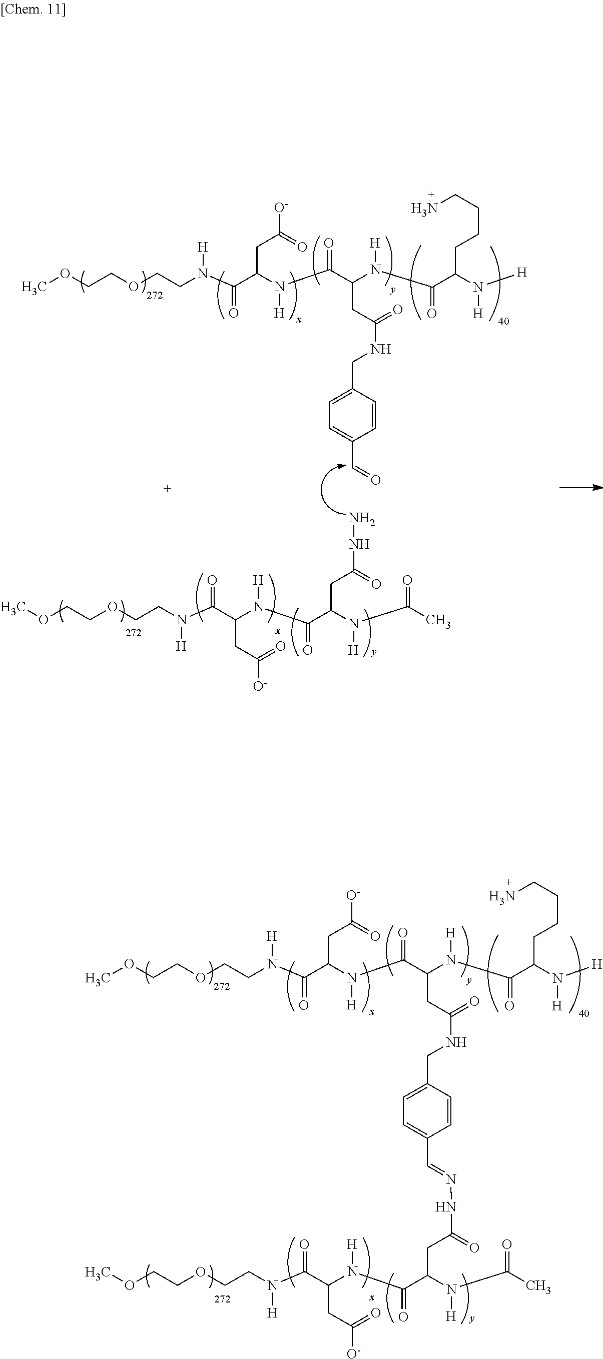

Alternatively, in the case where L² in Formula (II) is a group represented by the formula -L^{R}-NH—N═C(R¹)-L¹-NH—, the block copolymer represented by Formula (IIb-1) and the block copolymer represented by Formula (IIb-2) may be crosslinked with a linker to obtain the block copolymer represented by Formula (II).

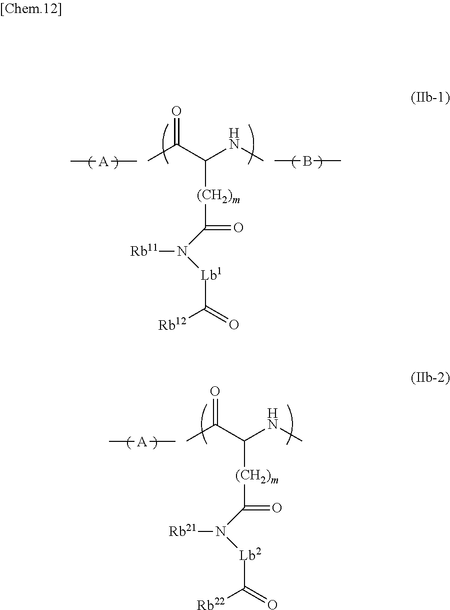

wherein A, B and m are the same as defined for A, B and m in Formula (I); Rb¹¹ and Rb²¹ represents a hydrogen atom; Lb¹ and Lb² represents an alkylene group, an arylene group or an aralkylene group, provided that Lb¹ and Lb² may have a substituent which is inactive with a hydrazide group or a hydrazine group; and Rb¹² and Rb²² represents a hydrogen atom, an aliphatic hydrocarbon group, or an aromatic hydrocarbon group.

Rb¹¹, Rb¹² and Lb¹ in Formula (IIb-1) are the same as defined for Ra¹¹, Ra¹² and La¹ in Formula (Ia-1), respectively.

Rb²¹, Rb²² and Lb² in Formula (IIb-2) are the same as defined for Ra¹¹, Ra¹² and La¹ in Formula (Ia-1), respectively.

An example of the alternative reaction scheme for producing the poly-ion complex micelle according to the present embodiment is shown below.

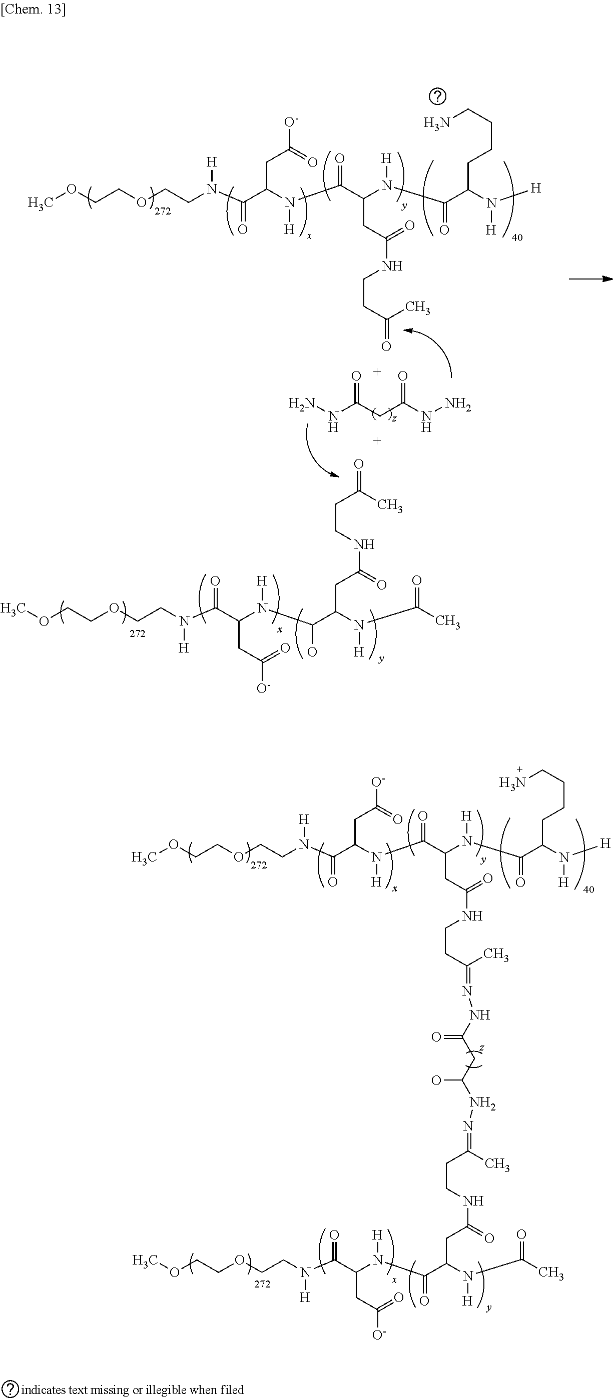

The poly-ion complex micelle may include one kind of block copolymer or two or more kind of block copolymers.

The poly-ion complex micelle may include one kind of anionic molecule drug or two or more kind of anionic molecule drugs.

The block copolymer included in the poly-ion complex micelle may be linked to a functional molecule. Examples of the functional molecules include targeting molecules for the delivery of the poly-ion complex micelle to a target site. Examples of the targeting molecules include specific binding molecules which can specifically bind to a particular molecule, such as peptides, antibodies or fragments thereof, and ligand molecules. The functional molecule may be linked to either or both of the terminal of the first hydrophilic block of the first block copolymer chain and the terminal of the second hydrophilic block of the second block copolymer chain. The functional molecule may be linked to the block copolymer by the conventional methods, such as click chemistry.

The poly-ion complex micelle according to the present embodiment described above includes a block copolymer having a hydrophilic block portion, a cationic hydrophobic block portion and a crosslinking block portion positioned between the hydrophilic block portion and the cationic hydrophobic block portion. As described above, since the crosslinking block portion surrounds the core loaded with the anionic molecule drug, and the hydrophilic block portion forms a shell, the core is stabilized. As a result, it becomes possible to encapsulate anionic molecule drugs stably. In particular, the poly-ion complex micelle according to the present embodiment may be applied to small anionic molecule drugs which is not possible to be stably encapsulated by conventional methods.

Further, the poly-ion complex micelle according to the present embodiment has the following advantages. Monodisperse particles, around 40-50 nm in size are formed, and most crosslinked micelles maintain narrow polydispersity in physiological saline compared to conventional non-crosslinked micelles. Further, the release rate of the drug in physiological saline is lower than conventional non-crosslinked micelles. Furthermore, polymer structure may be modified to impart different properties.

Furthermore, the poly-ion complex micelle according to the present embodiment has excellent cell membrane permeability, and therefore, it is possible to be efficiently taken up into a cell.

### EXAMPLES

The present invention will be described in detail based on the following examples. However, the embodiments of the present invention is not limited to the description of these examples.

### Synthesis Example 1: Synthesis of Triblock Co-Polymer, PEG-PBLA-PLys(TFA)

The synthesis of the triblock co-polymer, PEG-PBLA-PLys(TFA), was carried out by N-carboxyanhydride (NCA) ring-opening polymerization (ROP), as follows. The initiator in the first ROP step was α-Methoxy-ω-amino-poly(ethylene glycol) (Mw 12,000; PEG-NH₂), to produce the PEG-poly(β-benzyl L-aspartate) diblock co-polymer (PEG-PBLA). PEG-NH₂ was dried overnight in vacuo and dissolved in DMF. BLA-NCA (22 equivalents) was also dissolved in DMF then added to the PEG-NH₂ solution under Ar atmosphere, and then left to react at 35° C. for 72 h. The polymer was separated from the reaction mixture by precipitation in a mixture of n-hexane and ethyl acetate (6:4), followed by filtration and drying under vacuum.

The obtained PEG-PBLA was then used as initiator for the second ROP of Lys(TFA)-NCA to obtain PEG-PBLA-PLys(TFA). PEG-PBLA was dried overnight in vacuo and dissolved in DMSO. Lys(TFA)-NCA (40 equivalents) was also dissolved in DMSO then added to the PEG-PBLA solution under Ar atmosphere, and then left to react at 35° C. for 72 h. The triblock co-polymer was separated from the reaction mixture by precipitation in a mixture of n-hexane and ethyl acetate (6:4), followed by filtration and then finally drying under vacuum.

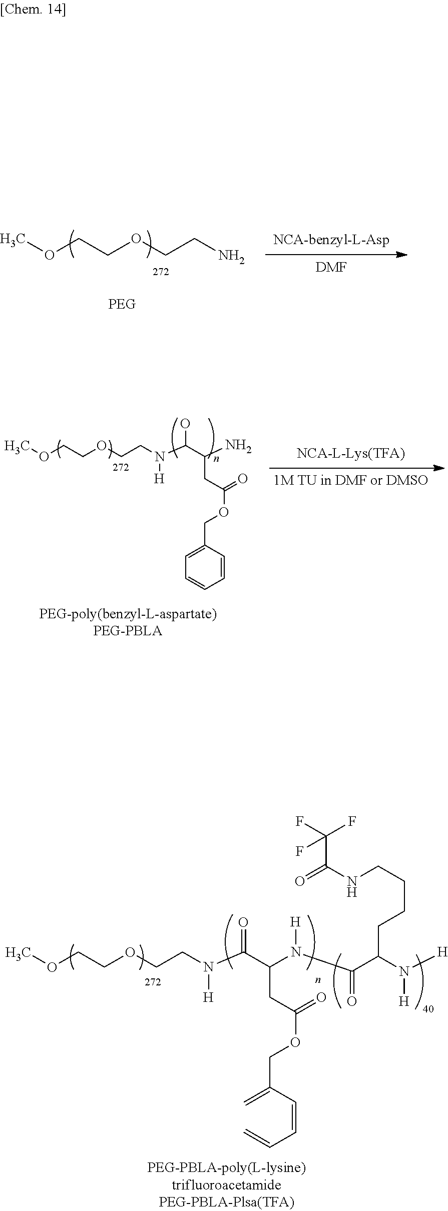

### Synthesis Example 2: Aminolysis of PEG-PBLA-PLys(TFA) and Deprotection

PEG-PBLA-PLys(TFA) (50 mg) was dissolved in DMF to which an aromatic aminoacetal linker, 1-[4-(dimethoxymethyl)phenyl]methanamine (30 eq) was added. The reaction mixture was stirred at 40° C. for 72 h. Thereafter, deprotection of the PLys(TFA) chain was carried out by adding 3 mL of methanol and 100 μL of 5 N NaOH. The reaction was allowed to proceed overnight. The mixture was then dialyzed against dilute acid and water for 48 h using a 7500 Da molecular weight cut off (MWCO) dialysis bag in which the dialyzing solution was changed 5 times. Dialysis against acid converts the acetal into an aldehyde functionality. The solution was freeze-dried under vacuum to obtain the modified triblock co-polymer ((PEG-PAsp(ArAld)-PLys)).

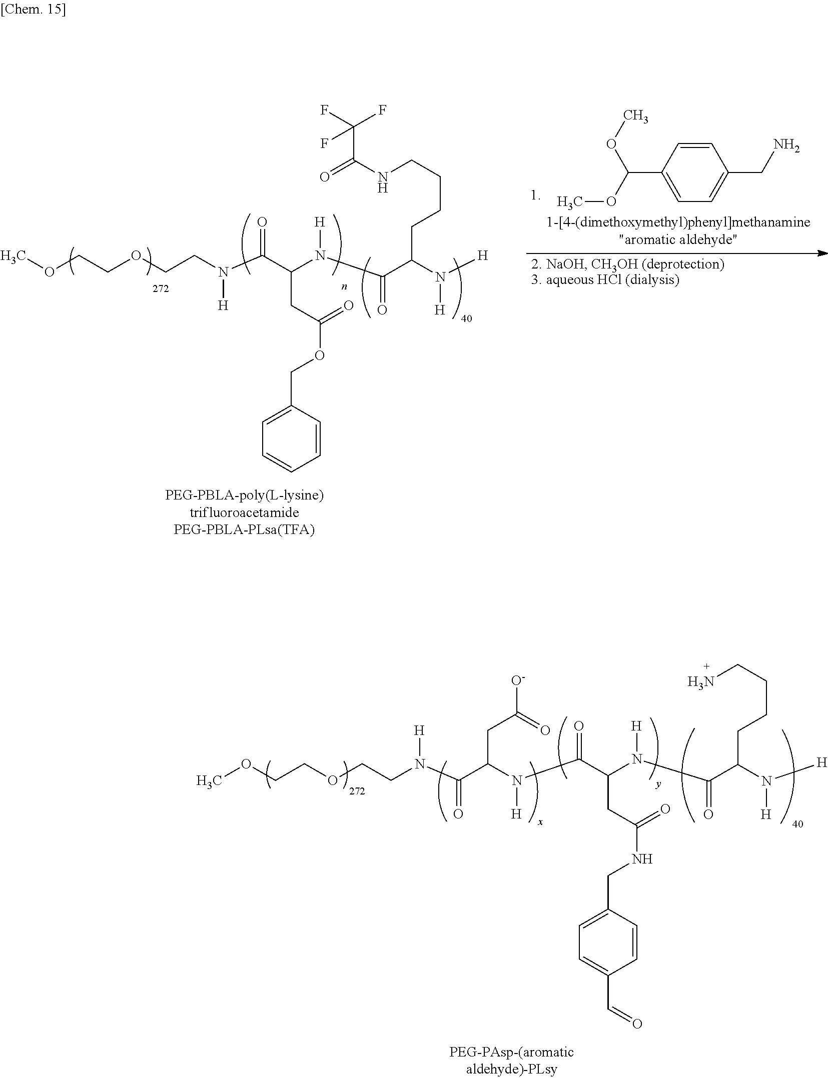

### Synthesis Example 3: Hydrazinolysis of PEG-PBLA-PLys(TFA) and Deprotection

Hydrazinolysis of PEG-PBLA-PLys(TFA) was carried out as follows. PEG-PBLA-PLys(TFA) (50 mg) was dissolved in DMF to which an excess of hydrazine monohydrate (50 μL) was added. The reaction mixture was stirred at 40° C. for 4 h. Thereafter, deprotection of the PLys(TFA) chain was carried out by adding 3 mL of methanol and 100 μL of 5 N NaOH. The reaction was allowed to proceed overnight. The mixture was then dialyzed against dilute acid and water for 48 h using a 7500 Da molecular weight cut off (MWCO) dialysis bag in which the dialyzing solution was changed 5 times. The solution was freeze-dried under vacuum to obtain the modified triblock co-polymer (PEG-PAsp(Hyd)-PLys).

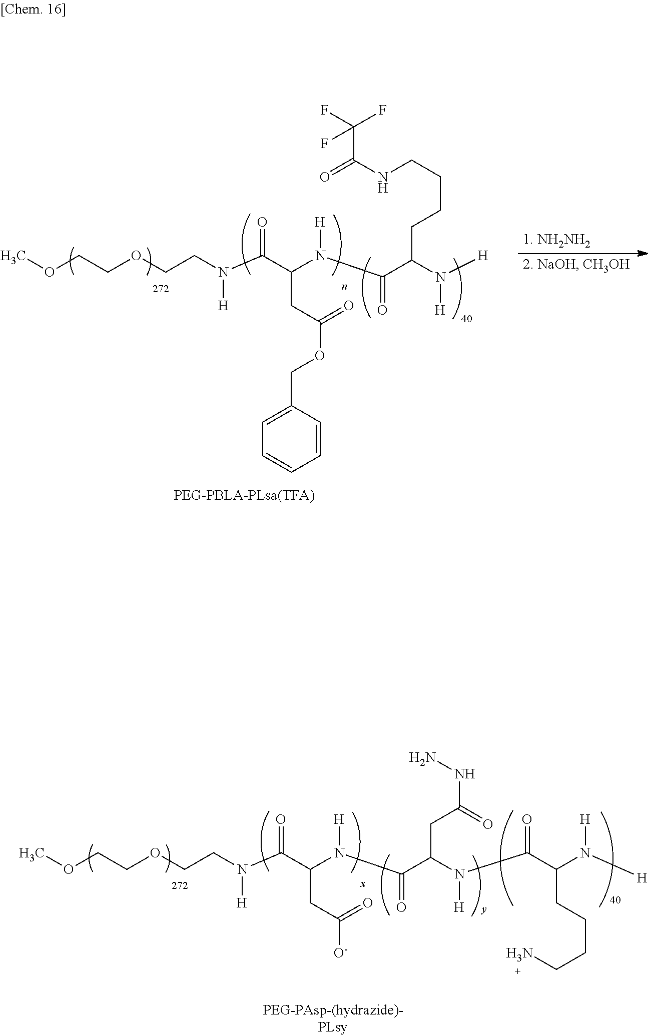

### Example 1: Preparation of Poly-Ion Complex Micelle (1)

Polymer solutions of triblock co-polymer PEG-PAsp(ArAld)-PLys (1) and triblock co-polymer (PEG-PAsp(Hyd)-PLys) were each dispersed in 10 mM phosphate buffer (PB) pH 5 at 2 mg/mL concentration. The resulting solutions were simply mixed with 1 mM gemcitabine triphosphate to attain a 1:1 cation-to-anion ratio, diluted with 10 mM PB pH 7.4 to desired concentration, and then vortexed. The micelle was allowed to cross-link at 4° C. for 48 h before passing through a 0.22-μM syringe filter. The crosslinked micelle (poly-ion complex micelle (1)) was formed by automatic self-assembly of polymers with the anionic drug cargo as shown in FIG. 1, and the hydrazone bond-formation occurred between the polymers as shown in the reaction scheme below.

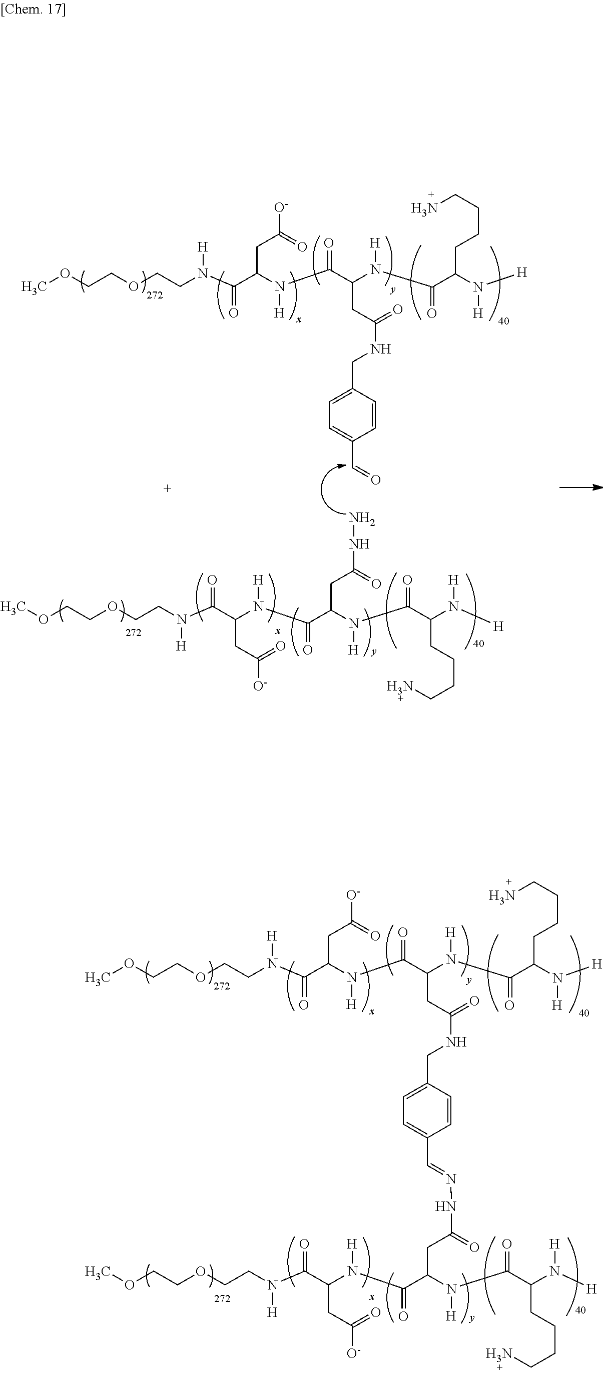

### Example 2: Preparation of Poly-Ion Complex Micelle (2)

The same procedure as in Example 1 was conducted, except that a triblock co-polymer PEG-PAsp(ArAld)-PLys (2) in which the chain length of PAsp(ArAld) was 12-22 repeating units was used instead of PEG-PAsp(ArAld)-PLys (1), so as to obtain poly-ion complex micelle (2).

### Example 3: Preparation of Poly-Ion Complex Micelle (3)

The same procedure as in Example 1 was conducted, except that a triblock co-polymer PEG-PAsp(aromatic ketone)-PLys (PEG-PAsp(ArKet)-PLys (1)) was used instead of triblock co-polymer PEG-PAsp(ArAld)-PLys, so as to obtain poly-ion complex micelle (3).

### Example 4: Preparation of Poly-Ion Complex Micelle (4)

The same procedure as in Example 3 was conducted, except that a triblock co-polymer PEG-PAsp(ArKet)-PLys (2) in which the chain length of PAsp(ArKet) was 12-22 repeating units was used instead of PEG-PAsp(ArKet)-PLys (1), so as to obtain poly-ion complex micelle (4).

### Example 5: Preparation of Poly-Ion Complex Micelle (5)

The same procedure as in Example 1 was conducted, except that cytarabine triphosphate was used instead of gemcitabine triphosphate, so as to obtain poly-ion complex micelle (5).

### Comparative Example 1: Preparation of Comparative Poly-Ion Complex Micelle (1)

A polymer solutions of diblock co-polymer PEG-PLys was dispersed in 10 mM phosphate buffer (PB) pH 5 at 2 mg/mL concentration. It was simply mixed with 1 mM gemcitabine triphosphate to attain a 1:1 cation-to-anion ratio, diluted with 10 mM PB pH 7.4 to desired concentration, and then vortexed. The micelle was formed by automatic self-assembly of polymers with the anionic drug cargo. The solution was then passed through a 0.22-μM syringe filter, so as to obtain comparative poly-ion complex micelle (1).

### Comparative Example 2: Preparation of Comparative Poly-Ion Complex Micelle (2)

The same procedure as in Comparative Example 1 was conducted, except that cytarabine triphosphate was used instead of gemcitabine triphosphate, so as to obtain comparative poly-ion complex micelle (2).

(Evaluation of Micelle Size and Polydispersity Index (1))

With respect to poly-ion complex micelle (1), poly-ion complex micelle (5), comparative poly-ion complex micelle (1) and comparative poly-ion complex micelle (2), the micelle size and the polydispersity index (PDI) were acquired using a dynamic light scattering (DLS) technique. The measurement conditions were as follows.

Temperature: 25° C., measurement angle: backscatter 173°, sample holder: quartz cuvette, automatic attenuator detection, no filter.

The results are shown in Table 1.

TABLE 1

| | | | Physiological | |
|---|---|---|---|---|
| | Water | | saline | |
| | | Poly- | | Poly- |
| | | dispersity | | dispersity |
| | Size (nm) | index | Size (nm) | index |
| Poly-ion | 45.4 ± 0.11 | 0.120 | 46.2 ± 0.28 | 0.108 |
| complex | | | | |
| micelle (1) | | | | |
| Poly-ion | 48.5 ± 0.30 | 0.147 | 46.1 ± 0.52 | 0.086 |
| complex | | | | |
| micelle (5) | | | | |
| Comparative | 35.8 ± 0.52 | 0.203 | Multiple populations | |
| poly-ion | | | detected | |
| complex | | | | |
| micelle (1) | | | | |
| Comparative | 38.2 ± 0.71 | 0.197 | Multiple populations | |
| poly-ion | | | detected | |
| complex | | | | |
| micelle (2) | | | | |

(Evaluation of Drug Release)

With respect to poly-ion complex micelle (1) and comparative poly-ion complex micelle (1), the drug release was evaluated as follows.

Micelle solutions were pipetted into Amicon Ultra-0.5 mL centrifugal filters (MWCO 10000) and spun (14000 g, 15 min, 4° C.). The filtrate was then collected, weighed, and then transferred into UV-transparent 96-well plates. Its UV absorption at 259 nm was measured using a microplate reader. Drug encapsulation was calculated by getting the ratio of the filtrate absorbance to that of the original (±)-C75-CoA solution added to form the micelle.

The results are shown in Table 2.

TABLE 2

| | % release in physiological saline |
|---|---|
| Poly-ion | 40 |
| complex | |
| micelle (1) | |
| Comparative | 80 |
| poly-ion | |
| complex | |
| micelle (1) | |

### Example 6: Preparation of Poly-Ion Complex Micelle (6)

The same procedure as in Example 1 was conducted, except that Luciferase ASO was used instead of gemcitabine triphosphate, so as to obtain poly-ion complex micelle (6).

### Comparative Example 3: Preparation of Comparative Poly-Ion Complex Micelle (3)

The same procedure as in Comparative Example 1 was conducted, except that Luciferase ASO was used instead of gemcitabine triphosphate, so as to obtain comparative poly-ion complex micelle (3).

<Evaluation of Micelle Size and Polydispersity Index (2)>

With respect to poly-ion complex micelle (6) and comparative poly-ion complex micelle (3), the micelle size and the polydispersity index (PDI) were evaluated in the same manner as in “Evaluation of micelle size and polydispersity index (1)”.

The results are shown in Table 3.

TABLE 3

| | Water | | Physiological saline | |
|---|---|---|---|---|
| | | Poly- | | Poly- |
| | | dispersity | | dispersity |
| | Size (nm) | index | Size (nm) | index |
| Comparative | 38.0 ± 0.56 | 0.064 | 39.3 ± 0.52 | 0.083 |
| poly-ion | | | | |
| complex | | | | |
| micelle (3) | | | | |
| Poly-ion | 42.6 ± 1.52 | 0.107 | 43.9 ± 0.04 | 0.068 |
| complex | | | | |
| micelle (6) | | | | |

(Evaluation of Cellular Uptake)

<Preparation of Crosslinked Poly-Ion Complex Micelle>

Polymer solutions of PEG-PAsp(ArAld)-PLys 12-22 (the chain length of PAsp(ArAld): 12-22 repeating units) and PEG-PAsp(Hyd)-PLys 12-22 (the chain length of PAsp(Hyd): 12-22 repeating units) were each dispersed in 10 mM phosphate buffer (PB) pH 5 at 10 mg/mL concentration. The solutions were simply mixed with 7.88 mM Fluor-CoA (Paraiso W K D, et al. Biomater. Sci. 9(21), 7076-7091 (2021)) to attain a 1:1 cation-to-anion ratio, diluted with 10 mM PB pH 7.4 to desired concentration, and then vortexed. The micelle was allowed to cross-link at 4° C. for 24 h before passing through a 0.22-μM syringe filter. The obtained crosslinked poly-ion complex micelle had a size of 44±0.4 nm, and the polydispersity index of 0.083.

<Preparation of DET Micelle>

Polymer solution of PEG-PAsp(DET) 12-69 was dispersed in 10 mM phosphate buffer (PB) pH 5 at 10 mg/mL concentration. The solution was simply mixed with 7.88 mM Fluor-CoA to attain a 1:1 cation-to-anion ratio, diluted with 10 mM PB pH 7.4 to desired concentration, and then vortexed. The solution was then passed through a 0.22-μM syringe filter.

<Evaluation Methods>

Cellular uptake of poly-ion complex micelles was evaluated by RPMI 2650 permeation model (Reichl S, Becker K. J. Pharm. Pharmacol. 64(11), 1621-1630 (2012)). Measurement of permeability coefficients of poly-ion complex micelles and Fluor-CoA was performed as reported in Gonzalez-Carter D, et al (J. Neuroendocrinol. 28(6) (2016)). RPMI 2650 (human nasal epithelia carcinoma, mucin-expressing cell) was used as cells.

<Results>

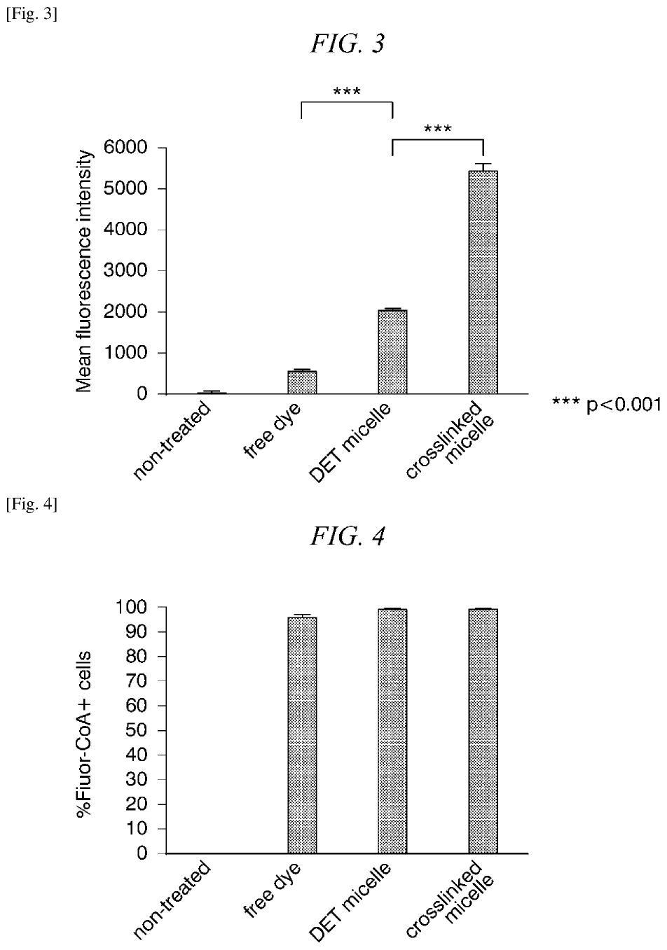

The results were shown in FIG. 3 and FIG. 4. As shown in FIG. 3, the crosslinked poly-ion complex micelle exhibited greater cellular uptake than DET micelle because of greater stability.

(Evaluation of Transwell Permeability)

<Preparation of Crosslinked Poly-Ion Complex Micelle>

The same procedure as described in [Evaluation of cellular uptake] was conducted, except that 4 kDa of FITC-Dextran was used instead of Fluor-CoA.

<Evaluation Methods>

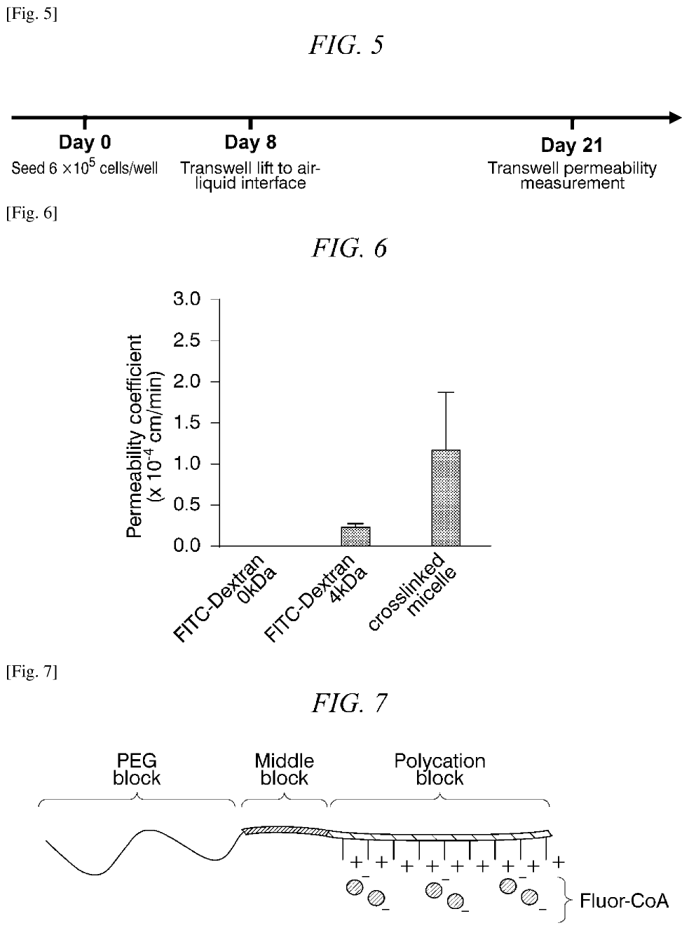

Culture of RPMI2650 using transwell was performed according to the timeline shown in FIG. 5. Transwell permeability was measured by the methods described in Reichl S, et al (J. Pharm. Pharmacol. 64(11), 1621-1630 (2012)).

<Results>

The results were shown in FIG. 6. FITC-Dextran 4 kDa exhibited minimal paracellular transport since it had low permeability. FITC-Dextran 70 kDa could not pass through the membrane at all. In contrast, the crosslinked poly-ion complex micelle exhibited good transwell permeability.

(Evaluation of Cellular Uptake into Brain Cells)

<Preparation of Crosslinked Poly-Ion Complex Micelle>

Preparation of PEG-PAsp(Hyd) 12-38 (the chain length of PAsp(Hyd):12-38 repeating units) was carried out according to the methods described in S, Cabral H, et al (J. Control. Release 188, 67-77 (2014)).

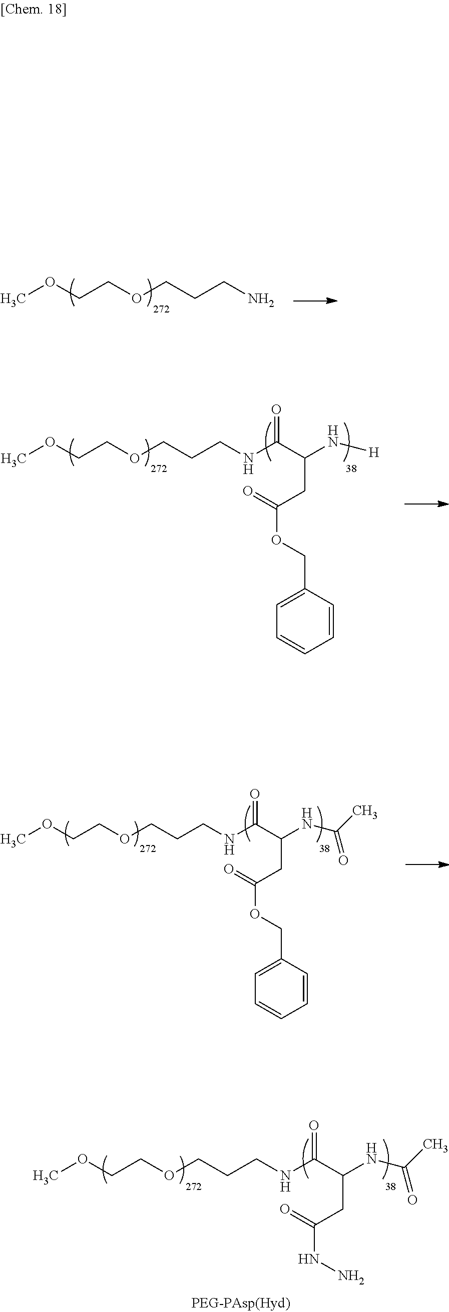

Polymer solutions of PEG-PAsp(ArAld)-PLys 12-22 and PEG-PAsp(Hyd) 12-38 were each dispersed in 10 mM phosphate buffer (PB) pH 5 at 10 mg/mL concentration. The solutions were simply mixed with 7.88 mM Fluor-CoA to attain a 1:1 cation-to-anion ratio, diluted with 10 mM PB pH 7.4 to desired concentration, and then vortexed. The micelle was allowed to cross-link at 4° C. for 24 h before passing through a 0.22-μM syringe filter. The obtained crosslinked poly-ion complex micelle had a size of 43±0.43 nm, and the polydispersity index of 0.13. FIG. 2 shows a schematic diagram of the crosslinked poly-ion complex micelle.

<Preparation of Non-Crosslinked Poly-Ion Complex Micelle>

Polymer solution of PEG-PAsp(ArAld)-PLys 12-22 was dispersed in 10 mM phosphate buffer (PB) pH 5 at 10 mg/mL concentration. The solution were simply mixed with 7.88 mM Fluor-CoA to attain a 1:1 cation-to-anion ratio, diluted with 10 mM PB pH 7.4 to desired concentration, and then vortexed. The solution was then passed through a 0.22-μM syringe filter. FIG. 7 shows a schematic diagram of the non-crosslinked poly-ion complex micelle. The obtained non-crosslinked poly-ion complex micelle had a size of 43±0.97 nm, and the polydispersity index of 0.13.

<Evaluation Methods>

The same procedure as described in [Evaluation of cellular uptake] was conducted, except that KT-5 (astrocytes), BV-2 (microglia), GT1-7 (nerons), or Rat primary brain endothelial cells were used instead of .RPMI 2650.

<Results>

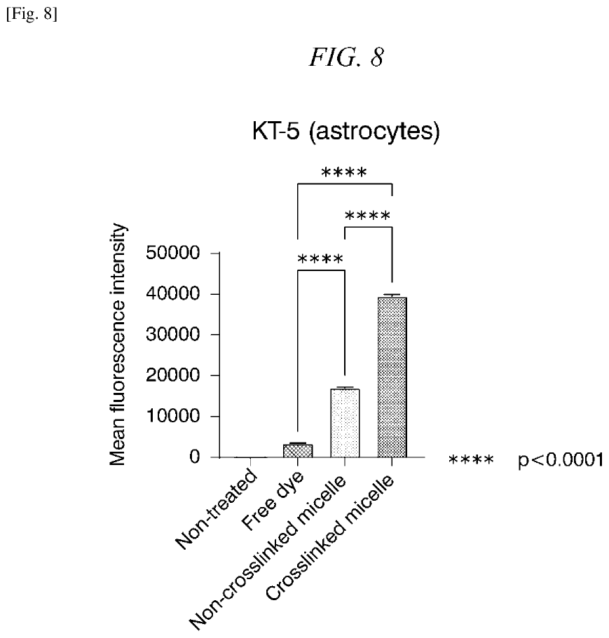

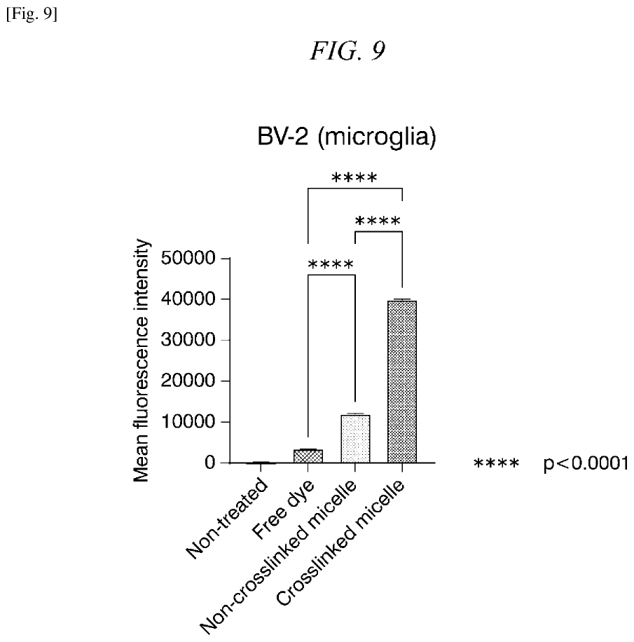

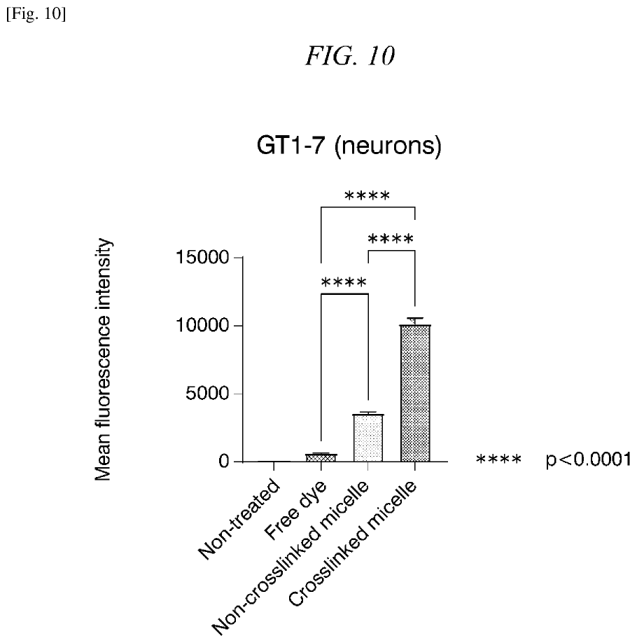

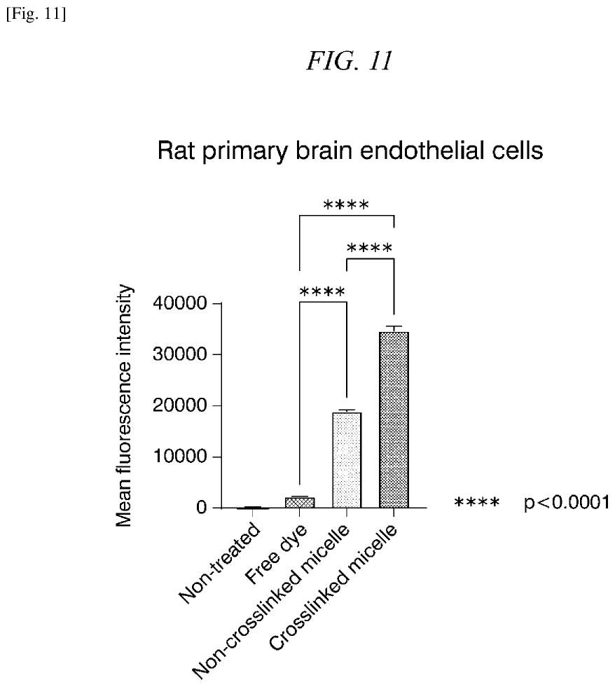

The results were show in FIGS. 8-11. In any cells, the crosslined poly-ion complex micelle was more efficiently taken up into the cells than the non-crosslinked poly-ion complex micelle.

(Preparation of Peptide-Conjugated Micelle)

The same procedure as described in [Evaluation of cellular uptake into brain cells] was conducted, except that azide-terminal PEG-PAsp(Hyd) 12-38 was used instead of PEG-PAsp(Hyd) 12-38 (which has methoxy-terminal). The click-conjugation of DBCO-linked peptide was done using the freeze-thaw method as described in Takemoto H, et al (Bioconjug. Chem. 23(8), 1503-1506 (2012)). Briefly, DBCO-peptide and Azide-terminal PEG-PAsp(Hyd) were mixed in equimolar concentrations and frozen at −30° C. for 8 h. The resulting peptide-conjugated polymer was thawed at 4° C. for 2 h and then dialyzed against ammonium bicarbonate buffer and freeze-dried to give the resulting polymer.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

1. A poly-ion complex micelle comprising:
a block copolymer having a hydrophilic block portion, a cationic hydrophobic block portion and a crosslinking block portion positioned between the hydrophilic block and the cationic hydrophobic block, and an anionic molecule drug encapsulated by the block copolymer,
wherein the crosslinking block has a hydrazone bond,
the block copolymer comprises the first block copolymer chain and the second block copolymer chain, the first block copolymer chain and the second block copolymer chain crosslinked to each other in the crosslinking block portion,
the hydrophilic block portion comprises the first hydrophilic block of the first block copolymer chain and the second hydrophilic block of the second block copolymer chain, and
the cationic hydrophobic block portion comprises the first cationic hydrophobic block of the first block copolymer chain.

2. The poly-ion complex micelle according to claim 1, wherein the cationic hydrophobic block portion further comprises the second cationic hydrophobic block of the second block copolymer chain.

3. The poly-ion complex micelle according to claim 2, wherein the block copolymer is represented by Formula (I): 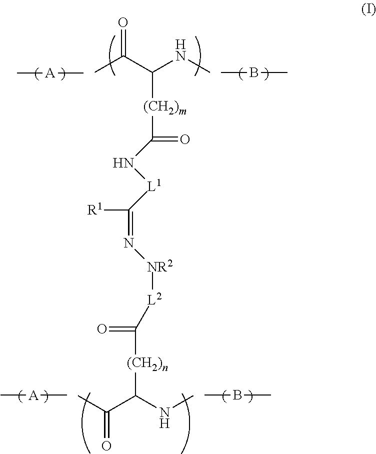
wherein A represents a repeating unit which constitutes the first hydrophilic block or the second hydrophilic block; B represents a repeating unit which constitutes the first cationic hydrophobic block or the second cationic hydrophobic block; m represents 1 or 2; L¹ represents a divalent linking group; R¹ represents a hydrogen atom, an aliphatic hydrocarbon group, or an aromatic hydrocarbon group; R² represents a hydrogen atom or a methyl group; L² represents a single bond or a divalent linking group; and n represents 1 or 2.

4. The poly-ion complex micelle according to claim 1, wherein the block copolymer is represented by Formula (II): 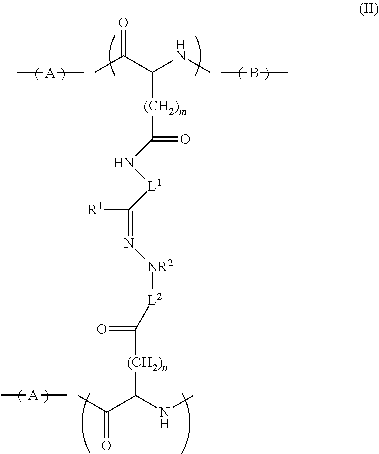
wherein A represents a repeating unit which constitutes the first hydrophilic block or the second hydrophilic block; B represents a repeating unit which constitutes the first cationic hydrophobic block; m represents 1 or 2; L¹ represents a divalent linking group; R¹ represents a hydrogen atom, an aliphatic hydrocarbon group, or an aromatic hydrocarbon group; R² represents a hydrogen atom or a methyl group; L² represents a single bond or a divalent linking group; and n represents 1 or 2.

5. The poly-ion complex micelle according to claim 1, wherein the cationic hydrophobic block is constituted of a repeating structure derived from polylysine.

6. The poly-ion complex micelle according to claim 1, which has a particle size of 20 to 100 nm, and a polydispersity index of 0.05 to 0.3.

7. The poly-ion complex micelle according to claim 1, wherein the anionic molecule drug has a net negative charge of −25 to −1 at physiological pH.

8. The poly-ion complex micelle according to claim 1, wherein the anionic molecule drug is a nucleic acid drug.
